(19) United States
(12) Patent Application Publication
(10) Pub. No.: US 2023/0005590 A1
(43) Pub. Date: Jan. 5, 2023

(54) METHOD FOR ALLOWING AND IMPROVING REHABILITATION AT HOME, VR REHABILITATION APPARATUS, AND SYSTEM EMPLOYING METHOD

(71) Applicants: HONG FU TAI PRECISION ELECTRONS (YANTAI) CO., LTD., Yantai (CN); HON HAI PRECISION INDUSTRY CO., LTD., New Taipei (TW)

(72) Inventors: CHENG-FA CHUNG, New Taipei (TW); KUN-LIN HUANG, New Taipei (TW); NA WANG, Yantai (CN); YU-MING JIAO, Shenzhen (CN)

(21) Appl. No.: 17/561,815

(22) Filed: Dec. 24, 2021

(30) Foreign Application Priority Data
Jul. 5, 2021 (CN) 202110757684.X

(51) Int. Cl. G16H 20/30; G16H 40/67; G16H 10/60

(52) U.S. Cl. CPC G16H 20/30; G16H 40/67; G16H 10/60

(57) ABSTRACT

A method for enabling rehabilitation at home for a user to regain a physical ability by means of an apparatus for physical use collects user information of a user. A virtual object is created based on the user information. The virtual object representing the user is displayed on a displaying mechanism. The virtual object is trained to move and perform rehabilitation actions. Actions and movements performed by the user are collected while the rehabilitation actions are displayed. The copycat actions of the user are compared with the rehabilitation actions and the user is assisted to correctly copy the rehabilitation actions based on the comparison. The user can undergo physiotherapy at home without a physiotherapist being present. A VR rehabilitation system and a VR rehabilitation apparatus applying the method are also provided in addition to the physical apparatus.

### FIELD

The subject matter herein generally relates to virtual reality (VR), specifically to method for rehabilitation using VR, a rehabilitation apparatus using VR, and a system employing the method.

### BACKGROUND

Rehabilitation is the process of regaining physical ability after the loss of the ability. The rehabilitation often is executed in a specified place, such as a hospital or a professional center, and each physical disability needs a physiotherapist for assisting. Having to travel to the specified place is inconvenient, and good physiotherapists are few in number. Rehabilitation teaching videos for standard use at home may not be suitable for every patient and so not effective in their purpose.

Thus, there is room for improvement in the art.

### BRIEF DESCRIPTION OF THE DRAWINGS

Implementations of the present disclosure will now be described, by way of example only, with reference to the attached figures.

FIG. 1 is a diagram illustrating an embodiment of a rehabilitation apparatus according to the present disclosure.

FIG. 2 is a diagram illustrating an embodiment of the rehabilitation apparatus in FIG. 1 according to the present disclosure.

FIG. 3 is a diagram illustrating an embodiment of a rehabilitation system according to the present disclosure.

FIG. 4 is a flowchart illustrating an embodiment of method for effective rehabilitation carried out at home according to the present disclosure.

### DETAILED DESCRIPTION

The present disclosure is described with reference to accompanying drawings and the embodiments. It will be understood that the specific embodiments described herein are merely part of all embodiments, not all the embodiments. Based on the embodiments of the present disclosure, it is understandable to a person skilled in the art, any other embodiments obtained by persons skilled in the art without creative effort shall all fall into the scope of the present disclosure.

The relationships of orientations or positions denoted by the terms of terms “center”, “longitudinal”, “lateral”, “length”, “width”, “thickness”, “up”, “down”, “left”, “right”, “horizontal”, “left”, “top”, “bottom”, “inside”, “outside”, “clockwise”, “anticlockwise” used herein refer to those illustrated in the accompany drawings, which are only for conveniently describing the invention and simplifying the description, rather than indicating or implying that a device or member has to be in a specific orientation or configured or operated in a specific orientation. In addition, the terms of “first” and “second” are for the purpose of describing only and should not be constructed to indicate or imply the relative importance. In the present disclosure, the term “some” means two or more than two, unless otherwise expressly stated.

In the present disclosure, unless otherwise expressly stated, the terms “mounted”, “link”, and “connect” should be understood broadly, unless otherwise specified and defined, for example, they may be a fixed connection or a removable connection, they may be mechanical connection or electrical connection, and also an inner communication between two members, they may be direct connection, and also an indirect connection via a medium, the skilled persons in the art may understand the meanings of above terms according to specific situations.

In the present disclosure, unless otherwise expressly stated, a structure in which a first feature is “on” or “below” a second feature may include an embodiment in which the first feature is in direct contact with the second feature and may also include an embodiment in which the first feature and the second feature are not in direct contact with each other but are in contact via an additional feature formed therebetween. Furthermore, a first feature “on,” “above,” or “on top of” a second feature may include an embodiment in which the first feature is right or obliquely “on,” “above,” or “on top of” the second feature, or just means that the first feature is at a height higher than that of the second feature; while a first feature “below,” “under,” or “on bottom of” a second feature may include an embodiment in which the first feature is right or obliquely “below,” “under,” or “on bottom of” the second feature, or just means that the first feature is at a height lower than that of the second feature.

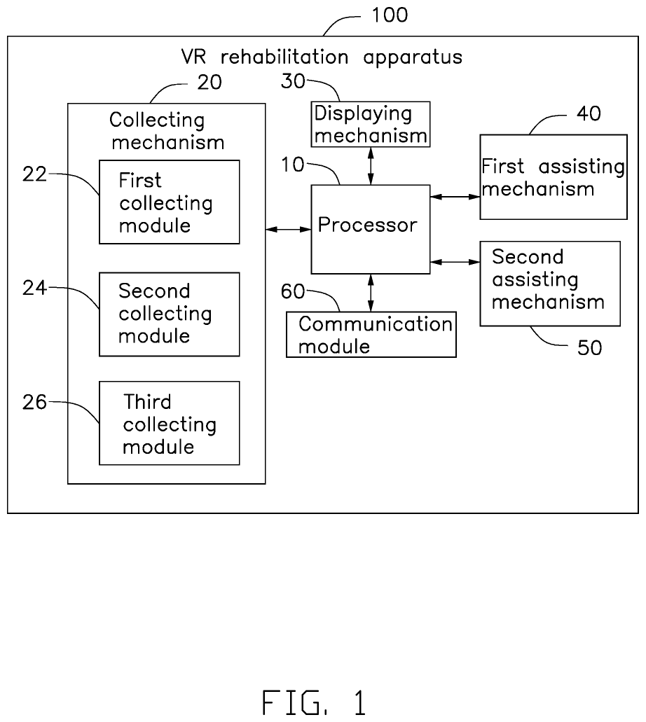

FIG. 1 shows an apparatus for rehabilitation at home making use of virtual reality (VR) principles (VR rehabilitation apparatus 100) for assisting users at home without a physiotherapist, thus no travel time of the user is required. The user can be someone with physical disabilities, or normal persons who want to improve their bodies or a part. The VR rehabilitation apparatus 100 includes a processor 10, a collecting mechanism 20, a displaying mechanism 30, and a first assisting mechanism 40. The processor 10 connects with the collecting mechanism 20, the displaying mechanism 30, and the first assisting mechanism 40.

The collecting mechanism 20 is configured to collect user information of the user and transmits the collected user information to the processor 10. The user information can include video data, user appearance features, and physiological data. The video data can record an action of each user in real time, for example, capturing a video of the user or his or her actions. The video data also can include environment information surrounding the user and sound information surrounding the user. The user appearance features are used for forming a virtual object corresponding to the user for making the virtual object more personal to the user. The physiological data can include a temperature, a heart rate, a heart rate variability of the user, not being limited. The physiological data can be provided to the processor 10, doctors, or physiotherapists to evaluate a rehabilitation for correcting or prompting.

The processor 10 is configured to create the virtual object based on the user information. The virtual object can be a 3D human model or a VR model. When the virtual object is the VR model, VR glasses are provided to the user for viewing a virtual object. In detail, the method for creating a virtual object of the embodiment can include collecting the user appearance by the collecting mechanism 20. The collecting mechanism 20 studies a body minutely based on a motion correction, and the processor 10 controls the displaying mechanism 30 to display the 3D body model. The processor 10 analyzes a skeletal structure of the 3D body model and generates joint node coordinates as a joint coordinate set. A vector is formed by two adjacent joints. The process 10 decides the joints to be tracked by the vector and confirms that the 3D body model can be used.

The processor 10 further controls the displaying mechanism 30 to display the virtual object. The displaying mechanism 30 can be a display screen or a projector. The displaying mechanism 30 can display the virtual object and generate a prompt sound. When the displaying mechanism 30 is a display screen, the displaying mechanism 30 can have a touch function, which can be touched by the users for inputting commands. When the displaying mechanism 30 is a projector, the displaying mechanism 30 projects the virtual object on a projection screen or other display surfaces.

The processor 10 also can train the virtual object to move and perform rehabilitation actions. The processor 10 trains the virtual object, thus the virtual object can portray the rehabilitation actions to form a rehabilitation video. Thus, the users can exercise according to the rehabilitation actions in the rehabilitation video.

The collecting mechanism 20 also collects actions acted by the users while the user mimes the rehabilitation actions and transmits the collected actions (mimicry actions) to the processor 10. While watching the rehabilitation video the user can mime and follow the actions acted by the virtual object. The collecting mechanism 20 can collect the mimicry actions of the user in real time.

The processor 10 further compares the mimicry actions with the rehabilitation actions to form a comparison result. The processor 10 compares the mimicry actions collected by the collecting mechanism 20 with the rehabilitation actions, thus the comparison result is formed. The comparison result can be displayed on the virtual object. For example, when the mimicry actions are similar or equal to the required rehabilitation actions, the rehabilitation actions acted by the virtual object are displayed in green for indicating correctness of the mimicry actions. When there is a difference between the mimicry actions and the rehabilitation actions, the rehabilitation actions acted by the virtual object are displayed in yellow for indicating incorrectness of the mimicry actions. When seeing the rehabilitation action acted by the virtual object in yellow, the user can re-mime the rehabilitation actions required. In detail, the method for forming comparison result can include: the processor 10 cooperates with the collecting mechanism 20 to capture coordinate of each joint based on the confirmed original position of the 3D body model. The vector between two joint nodes is used for determining whether the mimicry actions are correctly acted, thus the comparison result is formed.

The processor 10 further controls the first assisting mechanism 40 to assist the user for miming the rehabilitation actions based on the comparison result. When there is a difference between the mimicry actions and the rehabilitation actions, the processor 10 controls the first assisting mechanism 40 to assist the user to mime the rehabilitation actions. When the mimicry actions re-mimed by the user are incorrect for a certain number of times or in a certain time period, the processor 10 controls the first assisting mechanism 40 to assist the user for miming the rehabilitation actions. The first assisting mechanism 40 assists the user to mime the rehabilitation actions until the mimicry actions are similar or equal to the rehabilitation actions. It is understood that, when the mimicry actions are similar or equal to the rehabilitation actions, the body function of the user can be expected to recover.

The first assisting mechanism 40 assists the user to mime the mimicry actions. The processor 10 controls the first assisting mechanism 40 to assist the user based on a strength requirement of the rehabilitation actions. The processor 10 can omit the operation of comparing the mimicry actions with the rehabilitation actions.

If the body function of the user is improved or improving, the first assisting mechanism 40 can be omitted, the VR rehabilitation apparatus 100 can prompt the user based on the comparison result.

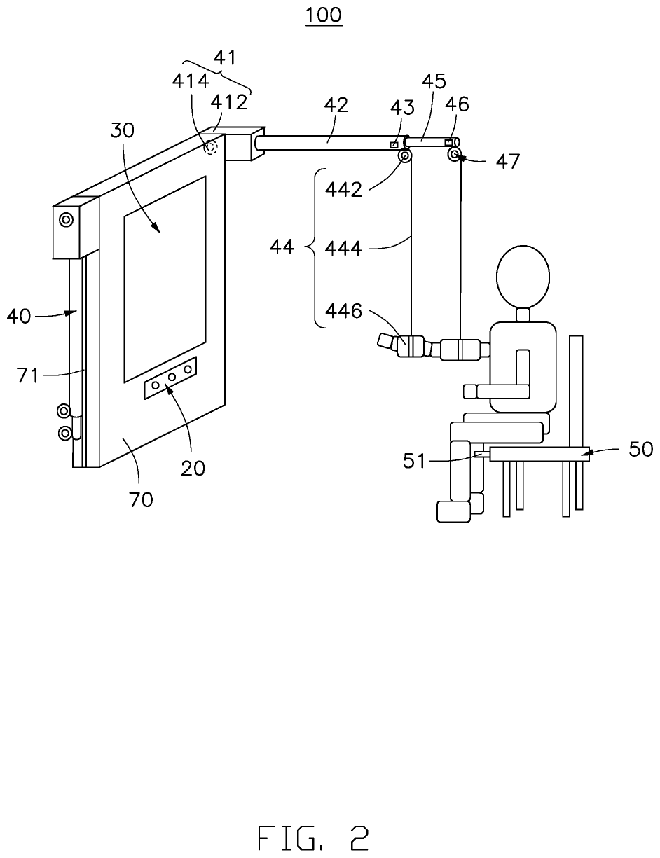

Referring to FIG. 2, in one embodiment, the VR rehabilitation apparatus 100 can include a frame 70. The processor 10 is disposed inside the frame 70. The collecting mechanism 20 is disposed inside the frame 70 or outside of the frame 70. The displaying mechanism 30 is disposed in the frame 70. The first assisting mechanism 40 can be slidably disposed on the frame 70.

The first assisting mechanism 40 can assist arm movements of the user to mime the mimicry actions. There is a first assisting mechanism 40 for each arm of the user. The two first assisting mechanisms 40 are slidably disposed on the frame 70.

The frame 70 includes two sliding rails 71 disposed on opposite sides of the frame 70. Each first assisting mechanism 40 includes a first driving module 41, a first arm 42, a second driving module 43, and a first assisting module 44. The first driving module 41 is slidably disposed on one of the sliding rails 71, the first arm 42 connects with the first driving module 41. The first driving module 41 drives the first arm 42 to move in a specified range. In detail, the first driving module 41 includes a first driving component 412 and a second driving component 414. The first driving component 412 connects with the second driving component 414, the second driving component 414 connects with the first arm 42.

The first driving component 412 and the second driving component 414 can be servo motors. The first driving component 412 drives the second driving component 414 and the first arm 42 to move along an extending direction of the sliding rail 71. The second driving component 414 drives the first arm 42 to rotate in a circle centered with the second driving component 414, thus the first arm 42 changes from a perpendicular state into a horizontal state. The first arm 42 can assist the user in providing a certain strength to the arms of the users in achieving certain movements.

In other embodiments, the first driving component 412 can be a cylinder, a linear motor module, and the like, which is able to drive the second driving component 414 and the first arm 42 to move along the extending direction of the sliding rail 71. The second driving component 414 can be an electromagnetic driving component, which is able to drive the first arm 412 to rotate.

The second driving module 43 is disposed on the first arm 42 or the frame 71. The first assisting module 44 is disposed on an end of the first arm 42 away from the first driving module 41. The second driving module 43 drives the first assisting module 44 to assist the user in following the mimicry actions.

In detail, the second driving module 43 includes an electromagnetic driving component. The first assisting module 44 includes a rotating component 442, an adjusting component 444, and an assisting component 446. The rotating component 442 can be a slide rail 71. The adjusting component 444 can be a rope. The assisting component 446 can be a bandage or a bracket, supporting the body of the user. The adjusting component 444 rolls on the rotating component 442. The assisting component 446 is disposed on an end of the adjusting component 444 away from the rotating component 442. The second driving module 43 connects with the rotating component 442 for driving the rotating component 442 to rotate, and the adjusting component 444 moves in a specified direction. Thus, the assisting component 446 assists the user to follow the mimicry actions. The adjusting component 444 circularly rolls on the rotating component 442 under the driving of the rotating component 442. The assisting component 446 moves towards to or away from the rotating component 442 for assisting the user to mime the mimicry actions.

For example, if a forearm of the user does not lift up at a specified position, the collecting mechanism 20 can collect the incorrect mimicry actions. The processor 10 compares the mimicry actions collected by the collecting mechanism 20 with the rehabilitation actions. The comparison result indicates there is a difference between the mimicry actions and the rehabilitation actions. The displaying apparatus 30 displays the rehabilitation action acted by the virtual object in yellow. After the user re-mimes the mimicry actions a certain number of times, the mimicry actions still being incorrect, the processor 10 can control the first assisting mechanism 40 to assist the user based on the comparison result. The second driving module 43 drives the assisting component 446 to drop down to the forearm. The user puts the forearm on the assisting component 446. Then, the second driving module 43 drives the assisting component 446 to lift up for assisting the user to mime the rehabilitation actions. In the above process, when the forearm is placed on the assisting component 446, the collecting mechanism 20 collects the placement, and the processor 10 controls the second driving module 43 to drive the assisting component 446 to be lifted up based on the placement. The user also can control the second driving module 43 to drive the assisting component 446 to be lifted up under a voice control, a remote control, and the like. Voice information and remote information can be collected by the collecting mechanism 20.

In one embodiment, the first assisting mechanism 40 further includes a second arm 45, a third driving module 46, and a second assisting module 47. The second arm 45, the third driving module 46, and the second assisting module 47 can assist an upper arm of the user.

The second arm 45 is slidably disposed on an end of the first arm 42 away from the first driving module 41. The third driving module 46 is disposed on the first arm 42 and the second arm 45. The second assisting module 47 is disposed on an end of the second arm 45 away from the first arm 42. The third driving module 46 drives the second assisting module 47 to cooperate with the first assisting module 44 for assisting the user to mime the rehabilitation actions. The structure of the third driving module 46 is similar to the structure of the second driving module 43. The third driving module 46 can be an electromagnetic driving component. The structure of the second assisting module 47 is similar to the structure of the first assisting module 44. The VR rehabilitation apparatus 100 can assist the forearm and the upper arm of the user to mime the rehabilitation actions by the first arm 42, the second arm 45, the corresponding driving modules, and the assisting modules.

In one embodiment, the VR rehabilitation apparatus 100 further includes a second assisting mechanism 50. The second assisting mechanism 50 assists legs of the user to mime the rehabilitation actions. The second assisting mechanism 50 is disposed at outer side of the frame 70. The second assisting mechanism 50 supports the user and assists the supported user to mime the rehabilitation actions.

In detail, the second assisting mechanism 50 is substantially in shape of a chair. The second assisting mechanism 50 can be a massage chair. The user can sit on the second assisting mechanism 50. The second assisting mechanism 50 includes a pulling module 51. The pulling module 51 corresponds to the leg of the user. The stretching and retracting pulling module 51 assists the legs of the user to mime the rehabilitation actions.

In other embodiments, the first assisting mechanism 40 can also assist the arms and the legs. The first assisting mechanism 40 includes the function of the second assisting mechanism 50, thus the second assisting mechanism 50 can be omitted.

In one embodiment, the collecting mechanism 20 includes a first collecting module 22, a second collecting module 24, and a third collecting module 26. The first collecting module 22 and the second collecting module 24 are disposed on the frame 70, and the third collecting module 26 is disposed on the outside of the frame 70. The third collecting module 26 can disposed on the second assisting mechanism 50, the first assisting mechanism 40, or the frame 70.

The first collecting module 22 includes an RGB color camera. The first collecting module 22 collects the mimicry actions of the user. The first collecting module 22 can capture videos, capture images, and collect sound around the user. When collecting the sound, the processor 10 can eliminate noise after comparing the sound for voice recognizing and locating a sound source. The videos captured by the first collecting module 22 can be stored, which is convenient to the remote doctors or physiotherapist for checking the rehabilitation state of the user and guiding the user to mime the rehabilitation actions. The second collecting module 24 includes an infrared emitter and an infrared 3D structure light range sensor, the second collecting module 24 collects the user appearance features and environment information. The environment information can include a distance between the user and the second collecting module 24 and a position of the user. The third collecting module 26 can be a body temperature sensor, a heart rate sensor, and the like, not being limited. The third collecting module 26 collects the physiological data of the user. The third collecting module 26 can be a wearable device, such as a bracelet, which is communicating with the processor 10 for collecting the physiological data of the user.

In one embodiment, the VR rehabilitation apparatus 100 further includes a communication module 60. The communication module 60 connects with the processor 10. The communication module 60 establishes a communication with an assisting authority. The communication module 60 can connect with an electronic device through a wireless manner, a BLUETOOTH manner, or a wired manner. The electronic device can include a specified application to connect with the communication module 60. The user communicates with the assisting authority through the communication module 60 and is able to make a video call or a voice call with the assisting authority.

The processor further controls the displaying mechanism 30 to display the information of the assisting authority. The information of the assisting authority can include a video information, a sound information, an operation instruction information generated by the assisting authority, a rehabilitation project, a scene, and resistance parameters selected by the assisting authority, not being limited. The assisting authority can teach the user through the video for miming the rehabilitation actions. The rehabilitation project selected and transmitted by the assisting authority can be displayed in the displaying mechanism 30 for being viewed by the user. The assisting authority can be a doctor or a physiotherapist, who can help the user to mime the rehabilitation train.

The processor 10 displays the video information and the sound information of the assisting authority on the displaying mechanism 30 for being viewed and learned by the user. The user can communicate with the assisting authority in real time. The assisting authority makes a video call with the user for watching the mimicry actions of the user. In one embodiment, the collecting mechanism 20 collects the mimicry actions of the user and transmits the mimicry actions to the assisting authority through the communication module 60. Or, the mimicry actions is packed by the processor 10, and the processor 10 transmits the packed mimicry actions to the assisting authority through the communication module 60 for watching and considering the mimicry actions of the user.

The processor 10 further generates a first instruction based on the information of the assisting authority. The assisting authority determines whether the mimicry actions of the user meet a standard. When the mimicry actions meet the standard, the assisting authority can be silent, and the user continues to mime the rehabilitation actions. When the mimicry actions do not meet the standard despite the user re-miming for the specified number of times, and the assisting authority generates instruction information based on the mimicry actions, the processor 10 generates the first instruction based on the instruction information generated by the assisting authority.

The processor 10 further controls the first assisting mechanism 40 and/or the second assisting mechanism 50 to assist the user to mime the rehabilitation actions based on the first instruction. In detail, the assisting authority transmits the instruction information when the watched mimicry actions do not meet the standard. The processor 10 generates the first instruction based on the instruction information and controls the first assisting mechanism 40 and/or the second assisting mechanism 50 to assist the user for miming the rehabilitation actions.

The processor 10 further can only control the second assisting mechanism 50 to assist the user for miming the rehabilitation actions.

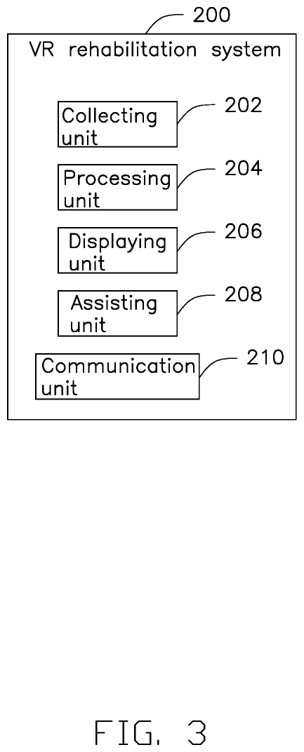

FIG. 3 shows a VR rehabilitation system 200. The VR rehabilitation system 100 can include one or more program instructions in form of applications stored in a storage medium, which can be executed by a processor to implement the functions of the present disclosure. In one embodiment, the VR rehabilitation system 200 includes a collecting unit 202, a processing unit 204, a displaying unit 206, and an assisting unit 208.

The collecting unit 202 is configured to collect user information of the user.

In detail, the collecting unit 202 can be the collecting mechanism 20 in FIG. 1. The collecting mechanism 20 includes a first collecting module 22, a second collecting module 24, and a third collecting module 26. The collecting mechanism 20 is configured to collect the user information and transmit the collected user information to the processing unit 204. The user information can include video data, user appearance features, and physiological data, not being limited.

The processing unit 204 is configured to create a virtual object based on the user information.

In detail, the processing unit 204 can be the processor 10 in FIG. 1. The processor 10 connects with the collecting mechanism 20. The processor 10 creates the virtual object based on the corresponding user information. The virtual object can be a 3D human model or a VR model.

The displaying unit 206 is configured to display a virtual object.

In detail, the displaying unit 206 can be the displaying mechanism 30 in FIG. 1. The displaying mechanism 30 connects with the processor 10. The displaying mechanism 30 can be a display screen or a projector. The displaying mechanism 30 can display the virtual object and generate a prompt sound.

The processor unit 204 is further configured to train the virtual object to move and perform rehabilitation actions.

In detail, the processor 10 also can train the virtual object to move and perform rehabilitation actions. The processor 10 trains the virtual object, thus the virtual object can move and perform the rehabilitation actions to form a rehabilitation video. Thus, the users can exercise according to the rehabilitation actions in the rehabilitation video.

The collecting unit 202 is further configured to collects mimicry actions acted by the users.

In detail, the collecting mechanism 20 collects the mimicry actions acted by the users and transmits the collected mimicry actions to the processor 10. The user exercises to mime the rehabilitation actions acted by the virtual object for forming the mimicry actions by watching the rehabilitation video. The collecting mechanism 20 collects the mimicry actions of the user in real time.

The processing unit 204 is further configured to compare the mimicry actions with the rehabilitation actions to form a comparison result.

In detail, the processor 10 compares the mimicry actions with the rehabilitation actions to form the comparison result. The processor 10 compares the mimicry actions collected by the collecting mechanism 20 with the rehabilitation actions, thus the comparison result is formed.

The assisting unit 208 is configured to assist the user to mime the rehabilitation actions based on the comparison result.

In detail, the assisting unit 208 can be the first assisting mechanism 40 and/or the second assisting mechanism 50 of FIG. 1. The first assisting mechanism 40 and/or the second assisting mechanism 50 connect with the processor 10. The processor 10 further controls the first assisting mechanism 40 and/or the second assisting mechanism 50 to assist the user to mime the rehabilitation actions based on the comparison result.

In one embodiment, the VR rehabilitation system 200 further includes a communication unit 210.

The communication unit 210 is configured to establish a communication with an assisting authority.

In detail, the communication unit 210 can be the communication module 60 in FIG. 1. The communication module 60 establishes the communication with the assisting authority. The user communicates with the assisting authority through the communication module 60 and is able to make a video call or a voice call with the assisting authority.

The displaying unit 206 is further configured to display information of the assisting authority.

In detail, the processor 10 controls the displaying mechanism 30 to display the information of the assisting authority.

The processing unit 204 is further configured to generate a first instruction based on the information of the assisting authority.

In detail, the processor 10 generates the first instruction based on the information of the assisting authority.

The assisting unit 208 is further configured to assist the user to mime the rehabilitation actions based on the first instruction.

In detail, the processor 10 controls the first assisting mechanism 40 and/or the second assisting mechanism 50 to assist the user for miming the rehabilitation actions based on the first instruction. When the mimicry actions watched by the assisting authority through the communication module 60 do not meet the standard, the assisting authority generates instruction information based on the mimicry actions, the processor 10 generates the first instruction based on the instruction information generate by the assisting authority. The processor 10 controls the first assisting mechanism 40 and/or the second assisting mechanism 50 to assist the user for miming the rehabilitation actions based on the first instruction.

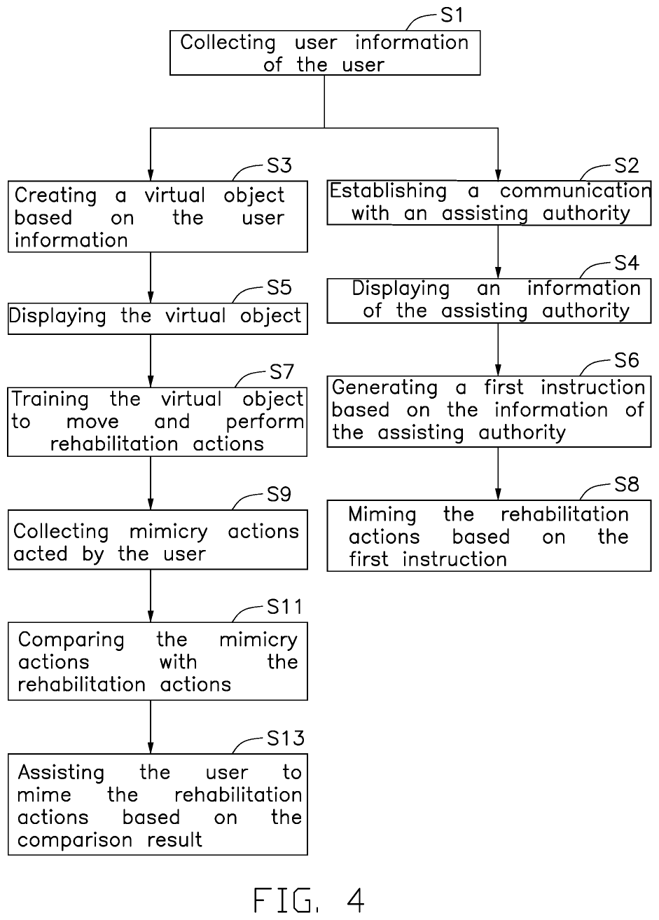

FIG. 4 shows a flowchart of a method for improving rehabilitation effect. Due to different requirements, a sequence of steps in the flowchart diagram can be changed, and some steps can be omitted. The method includes the following steps, these steps may be re-ordered:

In block S1, user information is collected.

In detail, the user information is collected by collecting unit 202. The collecting unit 202 can be the collecting mechanism 20 in FIG. 1. The collecting mechanism 20 is configured to collect the user information and transmit the collected user information to the processor 10. The user information can include video data, user appearance features, and physiological data, not being limited.

In block S3, a virtual object is created based on the user information.

In detail, the processing unit 204 creates the virtual object based on the user information. The processing unit 204 can be the processor 10 in FIG. 1. The processor 10 connects with the collecting mechanism 20. The processor 10 creates the virtual object based on the corresponding user information.

In block S5, the virtual object is displayed.

In detail, the virtual object is displayed on the displaying unit 206. The displaying unit 206 can be the displaying mechanism 30 in FIG. 1. The displaying mechanism 30 connects with the processor 10. The displaying mechanism 30 can be a display screen or a projector. The displaying mechanism 30 can display the virtual object and generate a prompt sound.

In block S7, the virtual object is trained to move and perform rehabilitation actions.

In detail, the virtual object is trained by the processing unit 204. The processing unit 204 can be the processor 10 in FIG. 1. The processor 10 trains the virtual object to move and perform the rehabilitation actions, thus the virtual object can move and perform the rehabilitation actions to form a rehabilitation video. Thus, the users can exercise according to the rehabilitation actions in the rehabilitation video

In block S9, mimicry actions acted by the users are collected.

In detail, the mimicry actions acted by the users are collected by the collecting unit 202. The collecting unit 202 can be the collecting mechanism 20 in FIG. 1. The collecting mechanism 20 collects the mimicry actions acted by the users and transmits the collected mimicry actions to the processor 10. The user exercises to mime the rehabilitation actions acted by the virtual object for forming the mimicry actions by watching the rehabilitation video. The collecting mechanism 20 collects the mimicry actions of the user in real time.

In block S11, the mimicry actions are compared with the rehabilitation actions to form a comparison result.

In detail, the mimicry actions are compared with the rehabilitation actions to form the comparison result by the processing unit 204. The processing unit 204 can be the processor 10 in FIG. 1. The processor 10 compares the mimicry actions collected by the collecting mechanism 20 with the rehabilitation actions, thus the comparison result is formed. The comparison result can be displayed on the virtual object.

In block S13, the user is assisted to mime the rehabilitation actions based on the comparison result.

In detail, the user is assisted by the assisting unit 208 to mime the rehabilitation actions based on the comparison result. The assisting unit 208 can be the first assisting mechanism 40 and/or the second assisting mechanism 50 of FIG. 1. The first assisting mechanism 40 and/or the second assisting mechanism 50 connect with the processor 10. The processor 10 further controls the first assisting mechanism 40 and/or the second assisting mechanism 50 to assist the user to mime the rehabilitation actions based on the comparison result.

In one embodiment, after the step of the block S1, the method further includes:

In block S2, a communication with assisting authority is established.

In detail, the communication with the assisting authority is established by the communication unit 210. The communication unit 210 can be the communication module 60 in FIG. 1. The communication module 60 establishes the communication with the assisting authority. The user communicates with the assisting authority through the communication module 60 and is able to make a video call or a voice call with the assisting authority.

In block S4, information of the assisting authority is displayed.

In detail, the information of the assisting authority is displayed by the displaying unit 206. The processor 10 controls the displaying mechanism 30 to display the information of the assisting authority. The information of the assisting authority can include a video information of the assisting authority, a sound information of the assisting authority, an operation instruction information generated by the assisting authority, a rehabilitation project, a scene, and resistance parameters selected by the assisting authority, not being limited. The assisting authority can be a doctor or a physiotherapist, who can help the user to move and perform the rehabilitation train. The processor 10 displays the video information and the sound information of the assisting authority on the displaying mechanism 30 for being viewed and learned by the user. The user can communicate with the assisting authority in real time. The assisting authority makes a video call with the user for watching the mimicry actions of the user.

In block S6, a first instruction is generated based on the information of the assisting authority.

In detail, the first instruction is generated by the processing unit 204 based on the information of the assisting authority. The assisting authority determines whether the mimicry actions of the user meet a standard. When the mimicry actions meet the standard, the assisting authority can be silent, and the user continues to mime the rehabilitation actions. When the mimicry actions do not meet the standard, the user re-mimes for the specified number of times, and the assisting authority generates instruction information based on the mimicry actions, the processor 10 generates the first instruction based on the instruction information generate by the assisting authority.

In block S8, the rehabilitation actions are mimed by the user based on the first instruction.

In detail, the user mimes the rehabilitation actions being assisting by the assisting unit 208 based on the first instruction. The assisting authority transmits the instruction information when the watched mimicry actions do not meet the standard. The processor 10 generates the first instruction based on the instruction information and controls the first assisting mechanism 40 and/or the second assisting mechanism 50 to assist the user for miming the rehabilitation actions.

A computer readable storage medium is provided in the present disclosure. The computer readable storage medium stores program codes, which can be executed by a processor to implement the above method for improving rehabilitation effect.

Based on the VR rehabilitation apparatus 100, the VR rehabilitation system 200, and the method for improving rehabilitation effect, the processor 10, the collecting mechanism 20, the displaying mechanism 30, the first assisting mechanism, the second assisting mechanism 50, and the communication module 60 are set for assisting the user to mime the rehabilitation actions at home without going to the hospital or the rehabilitation place. The displaying mechanism 30 displays the virtual object. The user can watch the virtual object to directly confirm own gesture and the mimicry actions for improving rehabilitation effect while using the VR rehabilitation apparatus 100. A psychological burden generated by the physical disabilities is decreased, and a confidence of the user can be improved. The collecting mechanism 20, the processor 10, the first assisting mechanism 40, and the second assisting mechanism 50 cooperate with each other for ensuring the user to correctly mime the rehabilitation actions acted by the virtual object. The processor 10 compares the mimicry actions with the rehabilitation actions and controls the first assisting mechanism 40 and/or the second assisting mechanism 50 to assist the user. Without the physiotherapist, the user also can correctly mime the rehabilitation actions by the assisted of the first assisting mechanism 40 and/or the second assisting mechanism 50. A correction of the mimicry actions and an exercise time and an intensity are ensured for recovering a body function.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A virtual reality (VR) rehabilitation apparatus comprises:
a processor connected with a collecting mechanism, a displaying mechanism, and a first assisting mechanism; and
the connecting mechanism configured to collect user information of an user, wherein:
the processor creates a virtual object based on the user information, controls the displaying mechanism to display the virtual object, and trains the virtual object to move and perform rehabilitation actions,
the collecting mechanism further collects mimicry actions acted while the user mimes the rehabilitation actions,
the processor compares the mimicry actions with the rehabilitation actions to form a comparison result, and
the processor controls the first assisting mechanism to assist the user based on the comparison result for correctly acting the rehabilitation actions.

2. The VR rehabilitation apparatus of claim 1, further comprising:
a frame, wherein:
the processor is disposed in the frame;
the collecting mechanism is disposed inside or outside the frame;
the displaying mechanism is disposed in the frame;
the first assisting mechanism is slidably disposed on the frame; and
the user information comprises video data, user appearance features, and physiological data.

3. The VR rehabilitation apparatus of claim 2, wherein:
two first assisting mechanisms are slidably disposed on the frame;
each of the two first assisting mechanisms comprises a first driving module, a first arm, a second driving module, and a first assisting module;
the first driving module is slidably disposed on one of the sliding rails;
the first arm connects with the first driving module;
the first driving module drives the first arm to move in a specified range;
the second driving module is disposed on the first arm;
the first assisting module is on an end of the first arm away from the first driving module; and
the second driving module drives the first assisting module to assist the user to mime the mimicry actions.

4. The VR rehabilitation apparatus of claim 3, wherein:
the second driving module comprises an electromagnetic driving component;
the first assisting module comprises a rotating component, an adjusting component, and an assisting component;
the adjusting component rolls on the rotating component;
the assisting component is disposed on an end of the adjusting component away from the rotating component; and
the second driving module connects with the rotating component for driving the rotating component to rotate and driving the adjusting component to move along a specified direction to assist the user to mime the mimicry actions.

5. The VR rehabilitation apparatus of claim 3, wherein:
the first assisting mechanism comprises a second arm, a third driving module, and a second assisting module;
the second arm is slidably disposed on an end of the first arm away from the first driving module;
the third driving module is disposed on the first arm and the second arm;
the second assisting module is disposed on an end of the second arm away from the first arm; and
the third driving module drives the second assisting module to cooperated with the first assisting module for assisting the user to mime the rehabilitation actions.

6. The VR rehabilitation apparatus of claim 2, further comprising:
a second assisting mechanism, wherein:
the second assisting mechanism is disposed outside the frame; and
the second assisting mechanism loads the user and assists the user to mime the rehabilitation actions.

7. The VR rehabilitation apparatus of claim 2, further comprising:
a first collecting module;
a second collecting module; and
a third collecting module, wherein:
the first collecting module, the second collecting module, and the third collecting module are disposed inside the frame or outside the frame,
the first collecting module collects the mimicry actions of the user,
the second collecting module collects user appearance features and environment information, and
the third collecting module collects the physiological data of the user.

8. The VR rehabilitation apparatus of claim 1, further comprising:
a communication module connected with the processor;
the communication module establishes a communication with an assisting authority; and
the processor further controls the displaying mechanism to display information of the assisting authority, generates a first instruction based on the information of the assisting authority, and controls the first assisting mechanism to mime the rehabilitation actions based on the first instruction.

9. A virtual reality (VR) rehabilitation system comprises a non-transitory storage medium with program codes, which when being executed by a processor, cause the processor to:
collect user information of an user;
create a virtual object based on the user information;
display the virtual object;
train the virtual object to move and perform rehabilitation actions;
collect mimicry actions acted by the user while miming the rehabilitation actions;
compare the mimicry actions with the rehabilitation actions to form a comparison result; and
assist the user to correctly mime the rehabilitation actions based on the comparison result.

10. The VR rehabilitation system of claim 9, wherein, the processor further performs:
establish a communication with an assisting authority;
display information of the assisting authority;
generate a first instruction based on the information of the assisting authority; and
assist the user to correctly mime the rehabilitation actions based on the first instruction.

11. The VR rehabilitation system of claim 9, wherein the user information comprises video data, user appearance features, and physiological data.

12. The VR rehabilitation system of claim 9, wherein the comparison result is displayed on the virtual object.

13. The VR rehabilitation system of claim 12, wherein when the mimicry actions are similar or equal to the rehabilitation actions, the rehabilitation actions acted by the virtual object are displayed in green for indicating correctness of the mimicry actions.

14. The VR rehabilitation system of claim 12, wherein when there is a difference between the mimicry actions and the rehabilitation actions, the rehabilitation actions acted by the virtual object are displayed in yellow for indicating incorrectness of the mimicry actions.

15. A method used in a VR rehabilitation apparatus; the VR rehabilitation comprises a storage medium with program codes, the program codes are executed by at least one processor to implement the following steps:
collecting user information of an user;
creating a virtual object based on the user information;
displaying the virtual object;
training the virtual object to move and perform rehabilitation actions;
collecting mimicry actions acted by the user while miming the rehabilitation actions;
comparing the mimicry actions with the rehabilitation actions to form a comparison result; and
assisting the user to correctly mime the rehabilitation actions based on the comparison result.

16. The method of claim 15, wherein the method further comprising:
establishing a communication with an assisting authority;
displaying information of the assisting authority;
generating a first instruction based on the information of the assisting authority; and
assisting the user to correctly mime the rehabilitation actions based on the first instruction.

17. The method of claim 15, wherein the user information comprises video data, user appearance features, and physiological data.

18. The method of claim 15, wherein the comparison result is displayed on the virtual object.

19. The method of claim 18, wherein when the mimicry actions are similar or equal to the rehabilitation actions, the rehabilitation actions acted by the virtual object are displayed in green for indicating correctness of the mimicry actions.

20. The method of claim 18, wherein when there is a difference between the mimicry actions and the rehabilitation actions, the rehabilitation actions acted by the virtual object are displayed in yellow for indicating incorrectness of the mimicry actions.
